(19) United States
(12) Patent Application Publication
(10) Pub. No.: US 2021/0353930 A1
(43) Pub. Date: Nov. 18, 2021

(54) APPARATUS AND METHOD FOR PROVIDING POSITION OF BRAIN STIMULATION

(71) Applicant: Neurophet Inc., Seoul (KR)

(72) Inventors: Dong Hyeon KIM, Seoul (KR); Sang Jin HAN, Seoul (KR)

(21) Appl. No.: 16/951,283

(22) Filed: Nov. 18, 2020

(30) Foreign Application Priority Data
May 15, 2020 (KR) 10-2020-0058280

(51) Int. Cl. A61N 1/04; G06T 7/00

(52) U.S. Cl. CPC A61N 1/0456; G06T 7/0012; G06T 2207/30016; G06T 2207/10088; A61N 1/0484

(57) ABSTRACT

The present disclosure relates to an apparatus and method for providing a position of brain stimulation in which the head shape of a patient is 3D modeled using Magnetic Resonance Imaging (MRI) for the brain of the patient, a stimulation target corresponding to a treatment location of the patient in the 3D modeled head shape of the patient is identified and selected, a plurality of optimal stimulation point candidates corresponding to a position of the selected stimulation target is designated, measurement simulations for each of the plurality of optimal stimulation point candidates are carried out so as to obtain measurement results, then one optimal stimulation point candidate showing a measurement result with utmost effect on the stimulation target is selected from the optimal stimulation point candidates.

### CROSS-REFERENCE TO RELATED APPLICATIONS

This application claims priority to and the benefit of Korean Patent Application No. 10-2020-0058280, filed in the Korean Intellectual Property Office on May 15, 2020, the contents of which are incorporated herein by reference in their entirety.

### BACKGROUND

### Technical Field

The present disclosure is related to a brain stimulation technology. In particular, the present disclosure is related to an apparatus and method for providing a position of brain stimulation which is used for an apparatus that is installed to a headgear for deep brain stimulation, transcranial electrical brain stimulation, etc., and releasing electric currents or ultrasonic waves, infrared rays, laser beams, electromagnetic waves, etc., toward the brain in order to provide treatments, operations, health improvement, etc.

### Related Art

Currently, various kinds of headgears for the treatment of patients suffering from brain diseases such as Parkinson's disease, memory disorders, depression, cerebral hemorrhage, etc., have been developed and then placed in commerce. Such a headgear is of an apparatus that releases electric currents, ultrasonic waves, infrared rays, laser beams, electromagnetic waves to a specific region of the brain of a patient for treatment or rehabilitation.

However, as shown in FIG. 1B, the structure of the human brain varies from individual to individual. Accordingly, even if providing stimulation to the same position as shown in FIG. 1A, a stimulation condition varies from individual to individual. Namely, since every person has a different brain structure, if providing stimulation following wearing a headgear of which a stimulation point is fixed, the strength of stimulation varies from individual to individual, thus reducing treatment effect.

Obviously, although using a headgear of which a stimulation point is changeable, it is necessary to identify optimal stimulation point for each person.

### SUMMARY

### Technical Problem

A technical object of the present disclosure is provided to an apparatus and method for providing a position of brain stimulation which identifies and provides the optimal stimulation point corresponding to the brain structure of a patient.

Further, a technical object of the present disclosure is provided to an apparatus and method for providing a position of brain stimulation which provides information for the optimal stimulation point for each patient corresponding to the structure of a wearable headgear, thus locating at least one stimulator installed to the headgear to the optimal stimulation point.

Technical objects to be achieved in the present disclosure are not limited to the aforementioned objects, and other objects will be understood from the following embodiments.

### Technical Solution

According to a first aspect of the present disclosure, an apparatus for providing a position of brain stimulation may include: a stimulation target selecting portion in which a head shape of a patient is 3D modeled using Magnetic Resonance Imaging (MRI) for a brain of the patient, and a position of a stimulation target corresponding to a treatment location from the 3D modeled head shape of the patient is selected; and a stimulation position selecting portion in which a plurality of optimal stimulation point candidates corresponding to the position of the stimulation target selected in the stimulation target selection portion is designated, and respective measurement simulations for each of the plurality of optimal stimulation point candidates are carried out so as to obtain measurement results, and one which shows a measurement result with utmost effect on the stimulation target is selected as an optimal stimulation point, from the candidates.

According to another aspect of the present disclosure, the apparatus for providing a position of brain stimulation may further include: a headgear information storing portion in which information for at least one headgear that is set on a head of the patient is stored and brain stimulation is provided through a stimulator; and a position adjustment information computing portion in which information for a headgear to be set on the patient is read from the headgear information storing portion and the read headgear information is applied to the 3D modeled head shape of the patient so as to compute stimulator adjustment information allowing the stimulator to correspond to the optimal stimulation point.

According to another aspect of the present disclosure, the stimulation target selecting portion may identify position information of the stimulation target for the patient using values of a table for the position information of the stimulation target set up according to a brain disease or a treatment location, and the stimulation target position may be found and selected in the 3D modeled head shape of the patient on the basis of the position information of the stimulation target.

According to another aspect of the present disclosure, the plurality of optimal stimulation point candidates may be patterned into a particular shape in the vicinity of the stimulation target.

According to another aspect of the present disclosure, the apparatus for providing a position of brain stimulation may further include an output portion representing input information on a screen, then outputting this information.

According to a second aspect of the present disclosure, a method for providing a position of brain stimulation may include following steps of: making a segmented area imaging by segmenting an area having different electrical feature in a Magnetic Resonance Imaging (MRI) of a first patient; 3D modeling a head shape of the patient using the segmented area imaging; identifying and selecting a position of a stimulation target corresponding to a treatment location of the patient in the 3D modeled head shape of the patient; designating a plurality of optimal stimulation point candidates corresponding to the selected position of the stimulation target; storing measurement results by carrying out respective measurement simulations for each of the plurality of optimal stimulation point candidates in order; identifying a first measurement result having utmost effect on the stimulation target by comparing the stored measurement results; and selecting, as an optimal stimulation point, an optimal stimulation point candidate corresponding to the first measurement result.

According to another aspect of the present disclosure, the method for providing a position of brain stimulation may further include following step of: computing stimulator adjustment information by applying information for a headgear to be set on the patient to the 3D modeled head shape of the patient, allowing a stimulator of the headgear to correspond to the optimal stimulation point.

According to another aspect of the present disclosure, in that in the step of identifying and selecting a position of the stimulation target, position information of the stimulation target for the patient may be identified using values of a table for the position information of the stimulation target set up according to a brain disease or a treatment location, and the stimulation target position may be found and selected in the 3D modeled head shape of the patient on the basis of the position information of the stimulation target.

### Advantageous Effects

According to the embodiment of the present disclosure, information for the optimal stimulation point for each patient or headgear adjustment information is provided, thereby locating a stimulator to the optimal stimulation point.

According to the embodiment of the present disclosure, it is capable of showing the highest treatment effect on each patient equally during the treatment using a headgear.

### BRIEF DESCRIPTION OF THE DRAWINGS

FIGS. 1A and 1B are views for explaining the extent of treatment effects corresponding to the brain structure of ordinary persons according to the same stimulation position.

FIG. 2 is a schematic view for explaining the concept of the present disclosure.

FIG. 3 is a schematic block diagram of an apparatus for providing a position of brain stimulation.

FIG. 4 is a flowchart showing a method for providing a position of brain stimulation according to an embodiment of the present disclosure.

FIG. 5 is a view showing a process for processing an MRI in the apparatus for providing a position of brain stimulation according to an embodiment of the present disclosure.

FIGS. 6A and 6B are views showing a stimulation target and optimal stimulation point candidates in a 3D brain imaging in the apparatus for providing a position of brain stimulation according to an embodiment of the present disclosure.

FIGS. 7A and 7B are views for a reference point to identify an initial position of the headgear used in the apparatus for providing a position of brain stimulation according to an embodiment of the present disclosure.

FIGS. 8A and 8B are views showing one example of the headgear used in the apparatus for providing a position of brain stimulation according to an embodiment of the present disclosure.

FIGS. 9A and 9B are views showing configurations of the key portions of the headgear used in the apparatus for providing a position of brain stimulation according to an embodiment of the present disclosure.

### DETAILED DESCRIPTION

Hereinafter, several embodiments will be described in detail referring to the accompanying drawings, so that a person having ordinary skill in the art (hereinafter, referred to as a ‘PHOSITA’) is able to implement the present disclosure easily. Further, the term “a portion” used in the specification may mean either a configuration element or a circuit.

Hereinafter, referring to accompanying drawings, an apparatus and method for providing a position of brain stimulation according to the present disclosure will be described.

FIG. 2 is a view for explaining the concept of the present disclosure. Referring to FIG. 2, the present disclosure identifies an optimal stimulation point suitable for the brain structure of a patient in a brain imaging of the relevant patient, then informing the patient or the expert of the identified optimal stimulation point.

Herein, the optimal stimulation point is of a point where stimulation (e.g., currents, ultrasonic waves, infrared rays, laser beams, electromagnetic waves, ultraviolet rays, etc.) that is provided to the brain of the patient through a stimulator of a headgear has the utmost effect on the patient. For example, referring to FIG. 2, when a first stimulation and a second stimulation are provided to a patient through a headgear 200, optimal stimulation points are a first position and a second position where the patient is affected at the utmost by the first stimulation and the second stimulation, respectively. When one stimulation is provided to the patient, one optimal stimulation point is provided. Similarly, when three stimulations are provided, three optimal stimulation points are provided.

Besides identifying an optimal stimulation point, the present disclosure generates and provides stimulator adjustment information allowing stimulation to be applied to the optimal stimulation point of the patient through the headgear 200 when the patient is wearing the headgear 200.

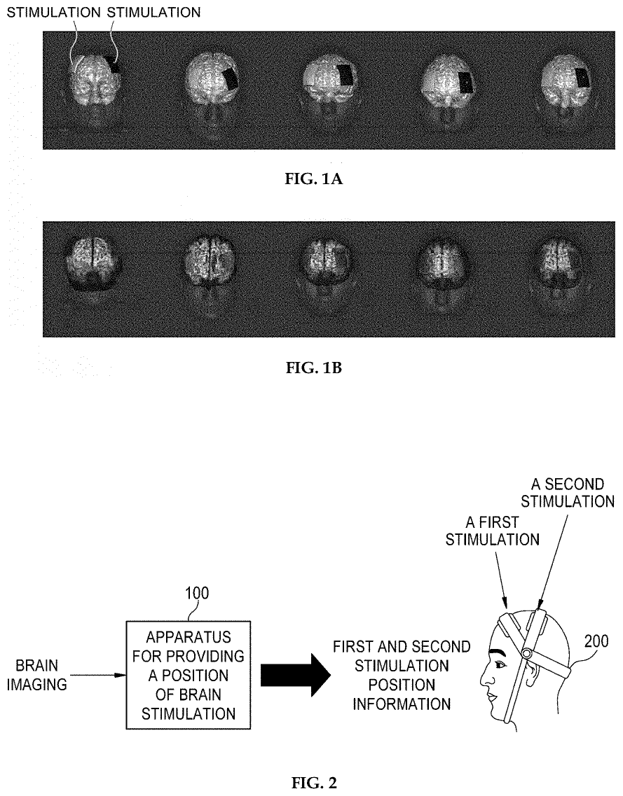

In general, although patients wear a headgear according to the correct wearing guide, it is the most likely that a position of a stimulator installed to the headgear for each patient is not located on the optimal stimulation point of the relevant patient. This is, as described referring to FIGS. 1A and 1B, because brain structures and head shapes vary from individual to individual.

Accordingly, following wearing a headgear, it is required for either the patient or the expert to adjust a position of the stimulator corresponding to the optimal stimulation point. At this time, stimulator position adjustment may be carried out according to eye measurement of the patient or the expert, thus causing a problem failing to locate the stimulator to the optimal stimulation point exactly.

The patient or the expert is capable of disposing the position of the stimulator to the optimal stimulation point by referring to the stimulator adjustment information. This stimulator adjustment information is favorable for either a headgear in which the stimulator is detachable or movable, or a headgear in which a part of a plurality of stimulators works during the treatment in a state that the plurality of stimulators is installed. However, this is not limited to the aforementioned headgears.

The present disclosure identifies and provides either the optimal stimulation point or stimulator adjustment information through a method for providing a position of brain stimulation or an apparatus for providing a position of brain stimulation 100.

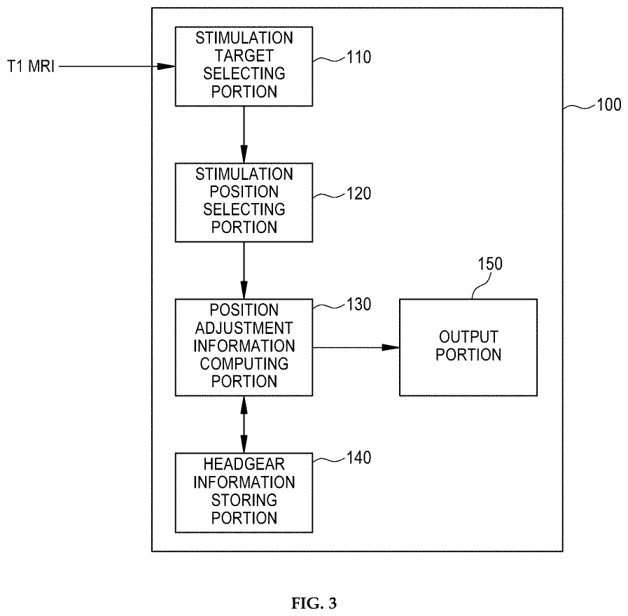

FIG. 3 is a schematic block diagram of an apparatus for providing a position of brain stimulation. Referring to FIG. 3, the apparatus for providing a position of brain stimulation 100 according to an embodiment of the present disclosure may include a stimulation target selecting portion 110 and a stimulation position selecting portion 120. Further, the apparatus for providing a position of brain stimulation 100 may further include a position adjustment computing portion 130 and a headgear information storing portion 140. The apparatus for providing a position of brain stimulation 100 may be configured to further include an output portion 150 when the output portion 150 is configured into an embedded form.

The stimulation target selecting portion 110 receives a Magnetic Resonance Imaging (MRI) for the brain of a patient, 3D modeling the head shape of the patient using the received MRI, identifying a position of a stimulation target from the 3D modeled head shape of the patient, and providing the identified position of the stimulation target to the patient or the expert through the output portion 150. Herein, the MRI may be one of T1 MRI or T2 MRI, wherein T1 MRI is more favorable than T2 MRI. T1 MRI is of an imaging representing a water component in black and a lipid component in white, while T2 MRI is of an imaging representing the water component in white and the lipid component in black.

The position of the stimulation target appears different according to a brain disease or a treatment location. Therefore, the stimulation target selecting portion 110 identifies a position designated by the expert as a position of the stimulation target, or identifying position information of the stimulation target for the relevant patient using values of a table for the position information of the stimulation target set up according to a brain disease or a treatment location, then finding and selecting the stimulation target in the brain imaging of the patient on the basis of the position information of the stimulation target. Herein, the position information of the stimulation target is not position information fixed in the brain imaging. This is of information that is capable of computing a position of the relevant stimulation target in a position of reference part fixed in the brain structure. The reference part may be one or more.

The stimulation position selecting portion 120 identifies a position of the stimulator, i.e., an optimal stimulation point which is capable of applying the utmost stimulation to a stimulation target among a plurality of stimulation positions corresponding to the position of the stimulation target identified in the stimulation target selecting portion 110, then providing the identified optimal stimulation point to the patient or the expert through the output portion 150.

The position adjustment information computing portion 130 reads information for a headgear to be set on the patient from information for headgears stored in the headgear information portion 140, applying the headgear information to the 3D modeled head shape of the patient, thus computing stimulator adjustment information allowing the stimulator of the headgear to correspond to the optimal stimulation point selected from the stimulation position selecting part 120. Herein, the headgear information may be information of either 3D headgear shape or numerical values for headgear shape, and position information for respective configurations.

The headgear information storing portion 140 stores information for at least one headgear.

The output portion 150 may be either a display device or a communication device of a mobile, a computer, etc. When output portion 150 is the communication device, the apparatus for providing a position of brain stimulation 100 further includes a communication module for the communication with the communication device.

Meanwhile, the stimulation target selecting portion 110, the stimulation position selecting portion 120, and the position adjustment information computing portion 130 may be either programs performing relevant functions or individual devices with the relevant programs. Herein, since PHOSITA will appreciate, as the general technology, the presence of a processor, such as CPU, etc., that controls the operation of the stimulation target selecting portion 110, the stimulation position selecting portion 120, and the position adjustment information computing portion 130, the description of the processor was left out.

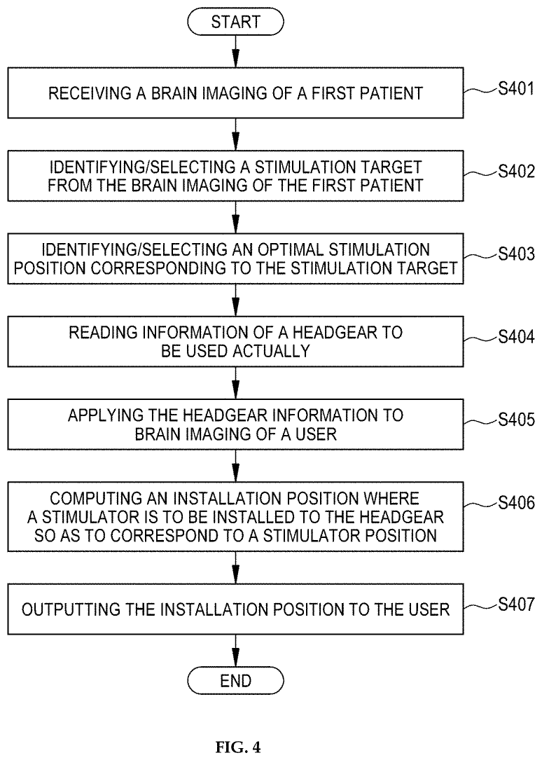

Hereinafter, referring to FIG. 4 to FIG. 6B, a method for providing a position of brain stimulation according to an embodiment of the present disclosure will be described.

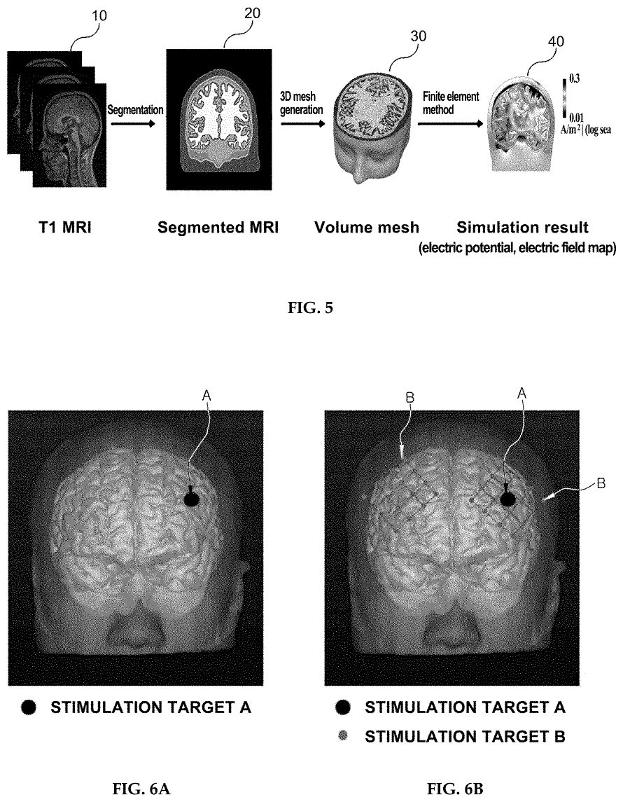

FIG. 4 is a flowchart showing a method for providing a position of brain stimulation according to an embodiment of the present disclosure, exemplifying the operation of the apparatus for providing a position of brain stimulation 100. FIG. 5 is a view showing a process for processing an MRI in in the apparatus for providing a position of brain stimulation according to an embodiment of the present disclosure. FIGS. 6A and 6B are views showing a stimulation target and optimal stimulation point candidates in a 3D brain imaging in the apparatus for providing a position of brain stimulation according to an embodiment of the present disclosure.

Firstly, the stimulation target selecting portion 110 receives T1 MRI 10 of an arbitrary patient (hereinafter, referred to as ‘a first patient’) or reads the same stored in a memory (not illustrated) (S401). The T1 MRI 10 is composed of a plurality of imaging frames.

Then, the stimulation target selecting portion 110 identifies and selects a stimulation target A using the T1 MRI 10 (S402).

Hereinafter, one example of S402 will be described in detail.

The stimulation target selecting portion 110 makes a segmented area imaging 20 by segmenting an area having different electrical feature in T1 MRI 10 of each frame. A segmentation reference complies with the following table 1 as known in general.

TABLE 1

| Area | Electrical Conductivity (unit: S/M) |
|---|---|
| White matter | 0.126 |
| Gray matter | 0.276 |
| Cerebrospinal fluid | 1.65 |
| Cranium | 0.01 |
| Skin | 0.465 |

The stimulation target selecting portion 110 combines the segmented area imaging of each of the frames, then generating a 3D brain imaging 30 composed of a plurality of Volumetric Meshes including triangular or tetragonal shapes.

In addition, a stimulation target is identified and selected in a state that the 3D brain imaging 30 is generated. At this time, in order to select the stimulation target, it is needed to obtain information for brain disease or treatment location of the first patient (hereinafter, referred to as ‘status information’). The expert is well-informed of this information, or that is stored in a memory (not illustrated) of the apparatus for providing a position of brain stimulation 100.

Two methods for selecting a stimulation target A are provided. One is referred to as ‘a first method’ accomplished by the expert and the other is referred to as ‘a second method’ accomplished in the stimulation target selecting portion 110.

According to the first method, once the expert designates a relevant target point in the 3D brain imaging 30 of the first patient by touching a screen or using input devices such as a mouse, a keyboard, etc., the stimulation target selecting portion 110 identifies and selects a position (i.e., coordinate) of the relevant target point in the 3D brain imaging 30 of the first patient.

The second method is accomplished in a state of storing values of a table for the position information of the stimulation target set according to a brain disease and a treatment location. Once the expert selects a brain disease or a treatment location of the first patient, the stimulation target selecting portion 110 identifies position information of a relevant stimulation target, then computing and selecting a stimulation target corresponding to the brain disease or treatment location of the first patient by using the identified position information of relevant stimulation target.

A position of the stimulation target A of the first patient, selected in the stimulation target selecting portion 110 is provided to the stimulation selecting portion 120, then being provided to the expert through the output portion 150 as shown in FIG. 6A. In FIG. 6A, a red spot indicated in the right brain of the first patient represents the stimulation target A.

Once receiving the stimulation target position, the stimulation position selecting portion 120 identifies and selects an optimal stimulation point corresponding to the stimulation target A (S403).

Hereinafter, S403 will be described in detail.

Once receiving the stimulation target position, the stimulation position selecting portion 120 designates a plurality of optimal stimulation point candidates patterned in the vicinity of the stimulation target A by using the position of stimulation target A as shown in FIG. 6B. The pattern of the plurality of optimal stimulation point candidates may be a tetragonal shaped matrix and this may be also a triangular, pentagonal and circular shape.

Since (FIG. 6B exemplifies one example that uses two stimulators providing anode and cathode currents, K (meaning any numbers) optimal stimulation point candidates are set corresponding to the stimulation target A of the right brain, and K optimal stimulation point candidates having the same pattern are also set in the left brain. It is favorable that the patterns of the optimal stimulation point candidates formed in the left and right brains are formed symmetrically based on a boundary line of the left and right brains.

In FIG. 6B, K means 16, however, it is not limited hereto.

Once the optimal stimulation point candidates are selected, the stimulation position selecting portion 120 finds at least one optimal stimulation point candidate providing the stimulation target A with the utmost stimulation when applying a stimulation to each of the optimal stimulation point candidates, respectively through a simulation using a stimulus intensity predictive technique, then identifying and selecting found, at least one optimal stimulation point candidate, as an optimal stimulation point. Herein, the number of at least one optimal stimulation point providing the stimulation target A with the utmost stimulation is the same as that of the stimulator used in the headgear 200.

For example, in a case of using two stimulators as shown in FIG. 6B, i.e., using the stimulators as an anode and a cathode, one optimal stimulation point candidate for the right brain and one optimal stimulation point candidate for the left brain are found as an electrode pair.

Therefore, when 16 optimal stimulation point candidates are set in each of the right and left brains respectively as shown in FIG. 6B, the stimulation position selecting portion 120 performs a simulation with one of the optimal stimulation point candidates in the left brain and one of the optimal stimulation point candidates in the right brain, as a pair. Accordingly, stimulations are performed total 256 times (16*16), storing every stimulation result of the stimulation target A for each simulation, comparing the respective simulation results, thus finding and selecting one electrode pair as the optimal stimulation point.

Hereinafter, the stimulus intensity estimation will be described as follows.

The stimulus intensity estimation performed in the stimulation position selecting portion 120 performs a simulation of the electric potential for electrical stimulation by using Governing equation, electrode position information, and stimulus intensity, wherein the Governing equation is drawn from Quasi-static Maxwell's equation of Equation 1 by using Finite Element Method (FEM) for 3D brain imaging 30 of the first patient composed of a plurality of Volumetric Meshes.

∇·(σ∇∇)=0 in Ω

n·J=0 on Ω (Equation 1)

In the above Equation 1, V is an electric potential, J is a current density, and Ω is a model of the head shape of the patient composed of 3D mesh.

Since the relationship between the electric potential obtained through the simulation in this manner and electric field is the same as following Equation 2, an electric field vector is calculated through 3D differentiation.

E=−∇v (Equation 2)

In the above Equation 2, V is an electric potential and E is electric field.

FIG. 5 shows one example of a brain imaging 40 for the electric current and electric field obtained through the simulation and differentiation in this manner.

Ultimately, the stimulation position selecting portion 120 finds an electrode pair that applies the utmost stimulation to the stimulation target A, then informing the expert of the found electrode pair as the optimal stimulation point through the output portion 150.

Meanwhile, once the expert is informed of the optimal stimulation point 150 through the stimulation position selecting portion 120, a treatment using the headgear 200 is then carried out. At this time, the expert adjusts a position of the stimulator installed to the headgear to be located to the optimal stimulation point in a state that the first patient is wearing the headgear 200.

Although such an adjustment behavior of the expert is carried out after identifying the optimal stimulation point through the output portion 150, this behavior is artificially done, thus resulting in an error between the stimulator position and the optimal stimulation point.

In order to solve this problem, the apparatus for providing a position of brain stimulation 100 according to the present disclosure may further include the position adjustment information computing portion 130 and the headgear information storing portion 140.

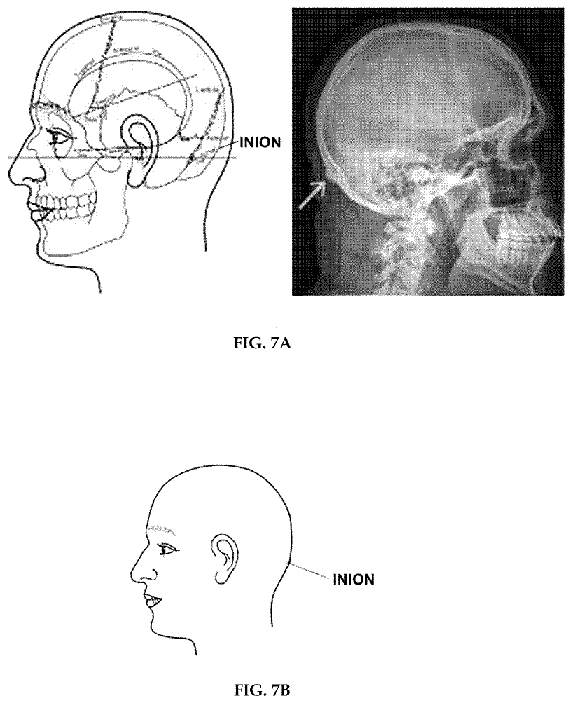

The position adjustment information computing portion 130 receives information for the optimal stimulation point from the stimulation position selecting portion 120, reading information of the headgear 200 to be set on the first patient from the headgear information storing portion 140 (S404), then applying this information to 3D brain imaging of the first patient (S405). At this time, a reference for applying information of the headgear 200 to the 3D imaging of the first patient is applied in the same fashion as a reference for setting the headgear 200 on the patient. Hereinafter, the reference for wearing the headgear 200 will be described referring to FIG. 7.

The position adjustment information computing portion 130 identifies the stimulator position in the headgear 200 at an initial position, identifying the optimal stimulation point corresponding to a relevant stimulator, then identifying a position error between the stimulator position and the optimal stimulation point or identifiable information of the stimulator located at the optimal stimulation point. Herein, the identification of the position error is of a type adopted for a headgear used by adjusting the stimulator position, while the identification of the stimulator identifiable information is of a type adopted for a headgear using a part of the plurality of stimulators.

In a case of the position error, the position adjustment information computing portion 130 changes the position error into moving position information up to the optimal stimulation point and/or installation position information on the basis of the present position of the stimulator (S406), then providing relevant information to the expert through the output portion 150 (S407). The moving position information is about moving the stimulator up to the optimal stimulation point, for example, moving in the forward direction at 30 degrees, a left direction at 50 degrees, etc. The installation position information is about informing a location of the stimulator to be installed in the headgear 200.

Hereinafter, one example for the use of information provided in the position adjustment computing portion 130 will be described, referring to FIGS. 7A to FIG. 9B.

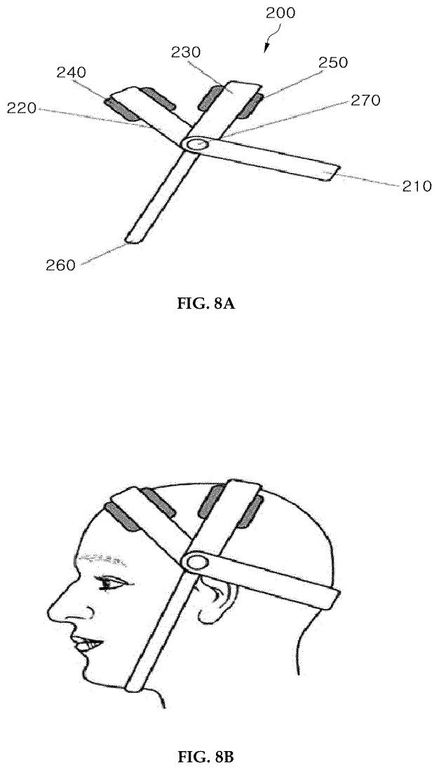

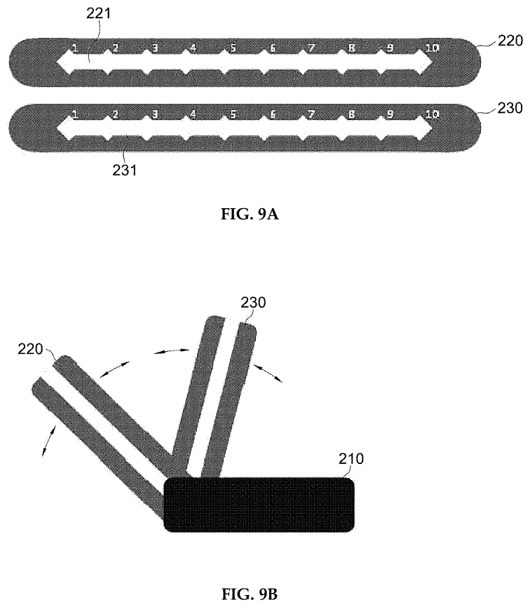

FIGS. 7A and 7B are views for a reference point in order to identify an initial position of the headgear used in the apparatus for providing a position of brain stimulation according to an embodiment of the present disclosure. FIGS. 8A and 8B show one example of the headgear used in the apparatus for providing a position of brain stimulation according to an embodiment of the present disclosure. FIGS. 9A and 9B show configurations of the key portions of the headgear used in the apparatus for providing a position of brain stimulation according to an embodiment of the present disclosure.

Referring to FIGS. 7A and 7B, an installation reference of the headgear is to locate a rear center of a wearing part in the headgear 200 to the inion, then hanging the headgear 200 on both ears. The application of the headgear 200 shown in FIGS. 8A and 8B exemplifies this installation reference.

Referring to FIGS. 8A and 8B, the headgear 200 is for transcranial electrical brain stimulation, including a wearing portion 210 to be set on the head circumference, a first adherence portion 220 to which a first stimulator 240 is adhered, a second adherence portion 230 to which a second stimulator 250 is adhered, the first stimulator 240 functioning as an anode, the second stimulator functioning as a cathode, and a chin strap 260.

Referring to FIGS. 9A and 9B, as shown in FIG. 9B, the first adherence portion 220 and the second adherence portion 230 are hinge-connected to the wearing portion 210 through a pivot joint 270, thus being configured rotatable. Further, as shown in FIG. 9A, the first adherence part 220 and the second adherence portion 230 have one slide groove respectively, and 10 coupling grooves are formed on the respective slide grooves, wherein the stimulators 240, 250 are coupled to the coupling grooves.

One example for the headgear wearing guide according to which the headgear 200 having these configurations is set on the patient and the position of the stimulator is adjusted to correspond to the optimal stimulation point, is as follows.

Firstly, the pivot joint part of the headgear 200 is located to be hung on both ears of the wearing object (patient). Secondly, the center of the wearing portion 210 is located to the inion of the wearing object. Thirdly, the first adherence portion 200 is adjusted as much as an angle of the moving position information computed in the position adjustment information computing portion 130. Fourthly, the second adherence portion 230 is adjusted as much as an angle of the moving position information computed in the position adjustment information computing portion 130.

Fifthly, the first stimulator 240 adhered to the first adherence portion 220 is moved from the pivot joint 270 as much as a distance of the moving position information computed in the position adjustment information computing portion 130. Sixthly, the second stimulator 250 adhered to the second adherence portion 230 is moved from the pivot joint 270 as much as a distance of the moving position information computed in the position adjustment information computing portion 130. Seventhly, the headgear 200 is firmly fastened using the chin straps 260.

The aforementioned descriptions are intended to provide exemplary configurations and operations for implementing the present disclosure. The technical scope of the present disclosure will include the aforementioned embodiments and embodiments obtainable by simply changing or modifying the above embodiments. Further, the technical scope of the present disclosure will include embodiments accomplishable by easily changing and modifying the aforementioned embodiments in further.

### FIGURE REFERENCE NUMBERS

100: an apparatus for providing a position of brain stimulation
200: a headgear
110: a stimulation target selecting portion
120: a stimulation position selecting portion
130: a position adjustment information computing portion
140: a headgear information storing portion
150: an output portion

1. An apparatus to provide a position of brain stimulation, the apparatus comprising:
a stimulation target selecting portion in which a head shape of a patient is 3D modeled using Magnetic Resonance Imaging (MRI) for a brain of the patient, and a position of a stimulation target corresponding to a treatment location from the head shape of the patient as 3D modeled is selected; and
a stimulation position selecting portion in which a plurality of optimal stimulation point candidates corresponding to the position of the stimulation target selected in the stimulation target selecting portion is designated, and respective measurement simulations for each of the plurality of optimal stimulation point candidates are carried out so as to obtain measurement results, and one optimal stimulation point candidate which shows a measurement result with utmost effect on the stimulation target is selected as an optimal stimulation point, from the plurality of optimal stimulation point candidates.

2. The apparatus to provide a position of brain stimulation according to claim 1, wherein the apparatus further comprises:
a headgear information storing portion in which information for at least one headgear that is set on a head of the patient is stored and brain stimulation is provided through a stimulator; and
a position adjustment information computing portion in which information for a headgear to be set on the patient is read from the headgear information storing portion and the read headgear information is applied to the 3D modeled head shape of the patient so as to compute stimulator adjustment information allowing the stimulator to correspond to the optimal stimulation point.

3. The apparatus to provide a position of brain stimulation according to claim 1, wherein the stimulation target selecting portion identifies position information of the stimulation target for the patient using values of a table for the position information of the stimulation target set up according to a brain disease or a treatment location, and the stimulation target position may be found and selected in the head shape of the patient as 3D modeled based on the position information of the stimulation target.

4. The apparatus to provide a position of brain stimulation according to claim 1, wherein the plurality of optimal stimulation point candidates is patterned into a particular shape in the vicinity of the stimulation target.

5. The apparatus to provide a position of brain stimulation according to claim 1, wherein the apparatus further comprises an output portion representing input information on a screen, then outputting this information.

6. A method of providing a position of brain stimulation, the method comprising:
making a segmented area imaging by segmenting an area having a different electrical feature in a Magnetic Resonance Imaging (MRI) of a first patient;
3D modeling a head shape of the patient using the segmented area imaging;
identifying and selecting a position of a stimulation target corresponding to a treatment location of the patient in the head shape of the patient as 3D modeled;
designating a plurality of optimal stimulation point candidates corresponding to the selected position of the stimulation target;
storing measurement results by carrying out respective measurement simulations for each of the plurality of optimal stimulation point candidates in order;
identifying a first measurement result having utmost effect on the stimulation target by comparing the stored measurement results; and
selecting, as an optimal stimulation point, an optimal stimulation point candidate corresponding to the first measurement result.

7. The method of providing a position of brain stimulation according to claim 6, wherein the method further comprises computing stimulator adjustment information by applying information for a headgear to be set on the patient to the shape of the patient as 3D modeled , allowing a stimulator of the headgear to correspond to the optimal stimulation point.

8. The method of providing a position of brain stimulation according to claim 6, wherein in identifying and selecting a position of the stimulation target, position information of the stimulation target for the patient is identified using values of a table for the position information of the stimulation target set up according to a brain disease or a treatment location, and the stimulation target position is found and selected in the head shape of the patient as 3D modeled based on the position information of the stimulation target.
